(19) United States
(12) Patent Application Publication
(10) Pub. No.: US 2010/0063592 A1
(43) Pub. Date: Mar. 11, 2010

(54) INTERLOCKING MULTI-PIECE INTERVERTEBRAL DISC PROSTHESIS AND METHOD FOR IMPLANTING SAME

(76) Inventors: James William Dwyer, Nashanic Station, NJ (US); James Ryan Dwyer, Nashanic Station, NJ (US)

Correspondence Address: KENYON & KENYON LLP, 1500 K STREET N.W., SUITE 700, WASHINGTON, DC, 20005, US

(21) Appl. No.: 12/205,032

(22) Filed: Sep. 5, 2008

(51) Int. Cl. A61F 2/44; A61B 17/04; A61B 17/58; A61B 19/00

(52) U.S. Cl. 623/17.16; 623/17.11; 606/74; 606/99; 128/898

(57) ABSTRACT

An interlocking intervertebral disc prosthesis is provided. The prosthesis can include a left prosthetic body and a right prosthetic body that interlock with one another. A kit for replacing an intervertebral disc with an interlocking intervertebral disc prosthesis is also provided. A method for surgically implanting an interlocking intervertebral disc prosthesis is also provided.

### TECHNICAL FIELD

The present invention generally relates to prostheses for replacing an intervertebral disc and methods for replacing the natural intervertebral disc. More specifically, the present invention relates to interlocking, multi-piece intervertebral disc prostheses.

### BACKGROUND

The intervertebral disc provides a mechanical cushion between adjacent vertebral segments of the spinal column and also maintains the proper anatomical separation between two adjacent vertebrae. This separation is necessary for allowing both afferent and efferent nerves to respectively exit and enter the spinal column. The disc also plays a key role in maintaining flexibility in the spinal column. In some instances, genetic or developmental irregularities, trauma, chronic stress, or degenerative disease can result in spinal pathologies necessitating removal of an intervertebral disc.

One option after removal of the intervertebral disc is completely replacing the natural disc with an artificial disc prosthesis. There are several designs of such total disc replacement prostheses, both for lumbar and cervical discs. One type of lumbar disc replacement includes an upper metal plate, a lower metal plate, and a polyethylene spacer positioned therebetween (for example, U.S. Pat. No. 6,645,248, incorporated herein by reference in its entirety). Many of these prostheses are impacted into the intervertebral disc space as a single component and require a rather invasive surgical procedure to implant. Other disc prostheses are and may be assembled intradiscally, but these devices usually comprise top and bottom pieces and still require relatively invasive surgery to impact their endplates. For example, in U.S. Pat. No. 6,726,720 issued to Ross et al. (incorporated herein by reference in its entirety), each of the top and bottom endplates may be impacted separately. However, this requires either an a difficult and invasive surgical approach (as shown in FIG. 7 of the Ross patent) or necessitates difficult maneuvering of the top-bottom components around sensitive dura matter.

Accordingly, in order to reduce highly invasive and difficult surgical procedures, there is a need for a different type of intervertebral disc prosthesis that can be implanted in a less invasive manner.

### SUMMARY

The present invention generally relates to an interlocking intervertebral disc prosthesis. In accordance with certain embodiments of the invention, a prosthesis comprises a left prosthetic body comprising at least one rigid endplate and a right prosthetic body comprising at least one rigid endplate. Each of the prosthetic bodies has a lateral surface, a medial surface, posterior surface, an anterior surface, a superior surface, and an inferior surface (all to be described in more detail below). The prosthesis additionally comprises means for interlocking the left prosthetic body to the right prosthetic body and means for accepting an insertion tool. In these embodiments, the interlocking means are located on the medial surfaces of the left and right prosthetic bodies, while the means for accepting an insertion tool are located on a surface of the left and right prosthetic bodies.

The present invention additionally provides an interlocking intervertebral disc prosthesis comprising a left prosthetic body comprising at least one rigid endplate and a right prosthetic body comprising at least one rigid endplate. Each of the prosthetic bodies has a lateral surface, a medial surface, a posterior surface, an anterior surface, a superior surface, and an inferior surface. A prosthesis in accordance with this embodiment additionally comprises at least one male fastener located on the medial surface of one of the left prosthetic body and the right prosthetic body and at least one female fastener located on the medial surface of the other of the left prosthetic body and the right prosthetic body. These male and female fasteners are configured and positioned such that the left prosthetic body and the right prosthetic body can interlock in an applied position of the prosthesis. A prosthesis in accordance with this embodiment additionally comprises one or more openings for accepting an insertion tool located on a surface of the right prosthetic body and a surface of the left prosthetic body.

The present invention also provides an interlocking intervertebral disc prosthesis comprising a left prosthetic body comprising at least one rigid endplate, a right prosthetic body comprising at least one rigid endplate, and at least one additional prosthetic body comprising at least one rigid endplate. Each of the left and right prosthetic bodies has a medial surface, a lateral surface, a posterior surface, an anterior surface, a superior surface, and an inferior surface. The at least one additional prosthetic body of this embodiment has a left surface, a right surface, a posterior surface, an anterior surface, a superior surface, and an inferior surface. In this embodiment, a prosthesis further comprises means for interlocking the at least one additional prosthetic to the left and right prosthetic bodies or any additional prosthetic bod(ies) that may be positioned between the left and right prosthetic bodies and that are, in turn, interlocked with the left and right prosthetic bodies. The interlocking means are located on the medial surfaces of the left and right prosthetic bodies and the left and right surfaces of the at least one additional prosthetic body. A prosthesis further comprises means for accepting an insertion tool located on a surface of each of the prosthetic bodies.

The present invention also provides a kit for replacing an intervertebral disc with an interlocking intervertebral disc prosthesis. In certain embodiments, the kit comprises an interlocking intervertebral disc prosthesis in accordance with embodiments of the present invention; a plurality of insertion tools attachable to the left and right prosthetic bodies, each insertion tool comprising an elongated member; and compression forceps comprising a set of handles and a pair of tongs pivotably attached to the set of handles.

The present invention also relates to a method for surgically implanting an interlocking intervertebral disc prosthesis. The method comprises removing the natural intervertebral disc from the intervertebral space and inserting a multi-piece prosthesis into the intervertebral space. The multi-piece prosthesis comprises a left prosthetic body comprising at least one rigid endplate and a right prosthetic body comprising at least one rigid endplate. The method further comprises interlocking the left prosthetic body and the right prosthetic body.

The present invention also provides an interlocking intervertebral disc prosthesis. The prosthesis of these embodiments comprises a left prosthetic body comprising at least one rigid endplate; a right prosthetic body comprising at least one rigid endplate, and means for interlocking the left prosthetic body to the right prosthetic body and accepting an insertion tool. Each of the prosthetic bodies of these embodiments has a lateral surface, a medial surface, a posterior surface, an anterior surface, a superior surface, and an inferior surface. The interlocking and accepting means are located on the medial surfaces of the prosthetic bodies of these embodiments.

The invention may be embodied in numerous devices and through numerous methods and systems. The following detailed description, taken in conjunction with the annexed drawings, discloses examples of the invention. Other embodiments, which incorporate some, all or more of the features as taught herein, are also possible.

### BRIEF DESCRIPTION OF THE DRAWINGS

The present invention will become more fully understood from the detailed description given hereinbelow and the accompanying drawings which are given by way of illustration only, and thus are not limitative of the present invention. Further, it should be noted that the illustrations are merely schematic and the various illustrated components can have different dimensions.

FIG. 1 is a posterior isometric view of a schematic illustration of an interlocking intervertebral disc prosthesis in accordance with certain embodiments of the present invention;

FIG. 2A is a posterior isometric view of a schematic illustration of a disc prosthesis according to an embodiment of the present invention illustrating some of the surfaces used to describe the disc prosthesis;

FIG. 2B is an anterior isometric view of a schematic illustration of a disc prosthesis according to an embodiment of the present invention illustrating other of the surfaces used to describe the disc prosthesis;

FIG. 3 is a posterior isometric view of a schematic illustration of an embodiment of a disc prosthesis of the present invention having left and right prosthetic bodies each comprising an inner core sandwiched between two rigid endplates;

FIG. 4 is a posterior isometric view of the prosthesis of FIG. 1 in an interlocked configuration (also referred to as an “applied position”);

FIG. 5A is a posterior isometric view of a schematic illustration of an exemplary left prosthetic body upper endplate having a generally arcuate profile;

FIG. 5B is a posterior isometric view of a schematic illustration of an exemplary left prosthetic body upper endplate having a generally domed or convex-like profile;

FIG. 5C is a posterior isometric view of a schematic illustration of an exemplary left prosthetic body upper endplate having a generally cylindrical profile;

FIG. 5D is a posterior isometric view of a schematic illustration of an exemplary left prosthetic body upper endplate having a generally rectangular profile;

FIG. 6A is an isometric view of a schematic illustration of an exemplary prosthetic body inner core having a generally cylindrical profile;

FIG. 6B is an isometric view of a schematic illustration of an exemplary prosthetic body inner core having a generally spherical or hemi-spherical profile;

FIG. 7 is a posterior isometric view of a schematic illustration of an embodiment of a disc prosthesis of the present invention having a plurality of serrations on the superior and inferior surfaces of the left and right prosthetic bodies;

FIG. 8 is a posterior isometric view of a schematic illustration of still another embodiment of a disc prosthesis of the present invention having three interlocking prosthetic bodies;

FIG. 9 is a posterior isometric view of an insertion tool and a schematic right prosthetic body in accordance with certain embodiments of the present invention;

FIG. 10 is an isometric view of a pair of compression forceps in relation to schematic left and right prosthetic bodies in accordance with certain embodiments of the present invention;

FIG. 11A is a top plan view of the pair of compression forceps of FIG. 10 in an open configuration and attached to schematic left and right prosthetic bodies in accordance with certain embodiments of the present invention;

FIG. 11B is a top plan view of a pair of compression forceps in a closed configuration and attached to schematic left and right prosthetic bodies in accordance with certain embodiments of the present invention

FIG. 12A is a posterior isometric view of a schematic left and right prosthetic body with an exemplary male fastener on the left prosthetic body according to an embodiment of the present invention;

FIG. 12B is a posterior isometric view of a schematic left and right prosthetic body with an exemplary male fastener on the left prosthetic body and an exemplary female fastener on the right prosthetic body according to an embodiment of the present invention;

FIG. 12C is a posterior isometric view of a schematic left and right prosthetic body with an exemplary male fastener on the left prosthetic body and an exemplary female fastener on the right prosthetic body according to an embodiment of the present invention;

FIG. 13A is a posterior view of a schematic left and right prosthetic body in communication with each other with an example of several male fasteners of various configurations on the left prosthetic body and several female fasteners of various configurations on the right prosthetic body;

FIG. 13B is a posterior view of a schematic left and right prosthetic body in communication with each other having both exemplary male and female fasteners on both the left and right prosthetic bodies;

FIG. 14 is a posterior isometric view of a schematic illustration of an exemplary left prosthetic body upper endplate having several pores extending from the superior surface to the inferior surface of the endplate;

FIG. 15 is an isometric view of an exemplary box chisel in accordance with certain embodiments of the present invention; and

FIG. 16 is a top plan view of a method in accordance with certain embodiments of the present invention, depicting a compression tool attached to insertion rods attached to two schematic illustrations of prosthetic bodies.

### DETAILED DESCRIPTION

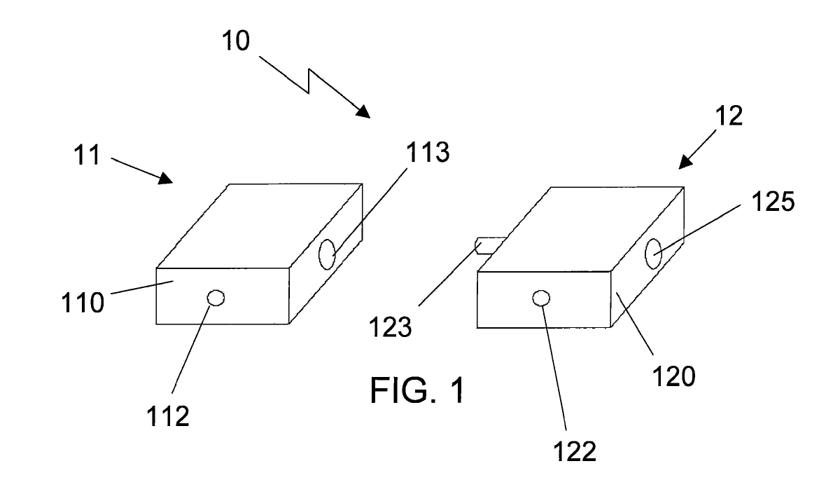

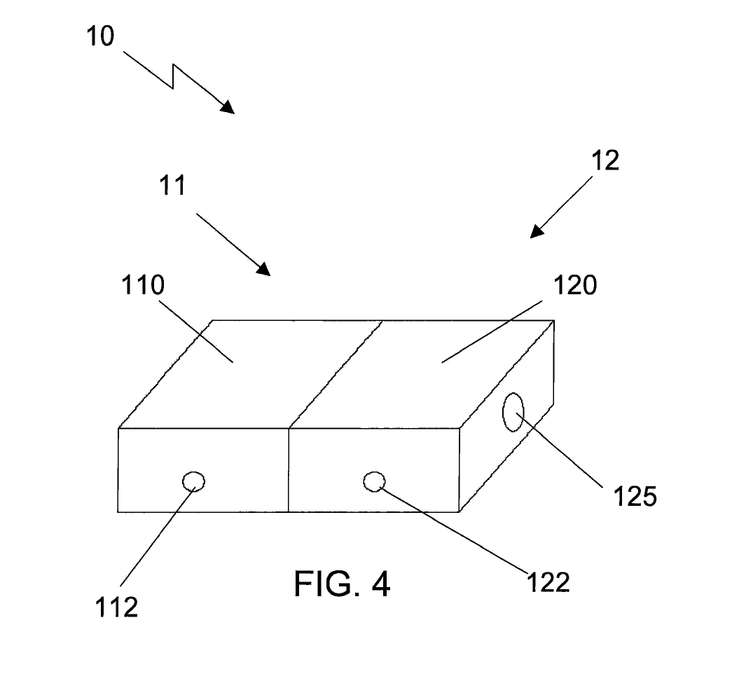

The present invention generally relates to multi-piece interlocking intervertebral disc prostheses. A disc prosthesis of the present invention interlocks in a left to right fashion as opposed to a top to bottom fashion. By “multi-piece” is meant that the prosthesis comprises separate pieces that can be assembled into a unit in an applied position of the prosthesis (i.e. when the prosthesis is implanted in the intervertebral space prior to just prior to closing the incision site, a schematic illustration of which is provided by FIG. 4). Likewise, multi-piece also means that, in a resting position, pieces of the prosthesis can be separated from other pieces without disrupting the integrity (i.e. breaking) the prosthesis. A “resting position” refers to the position of the prosthesis when in a disassembled state and placed on a flat table (such as shown in FIG. 1). As used herein, the terms “inferior,” “superior,” “anterior,” “posterior,” “medial,” “lateral,” “upward,” “downward,” “top,” “bottom,” “horizontal,” “vertical,” “left,” and “right,” or vocabular modifications of such terms as well as other directional or anatomical orientation terms refer to positions and configurations of a disc replacement in an applied position when the patient is in the anatomical position (a term well known in the art).

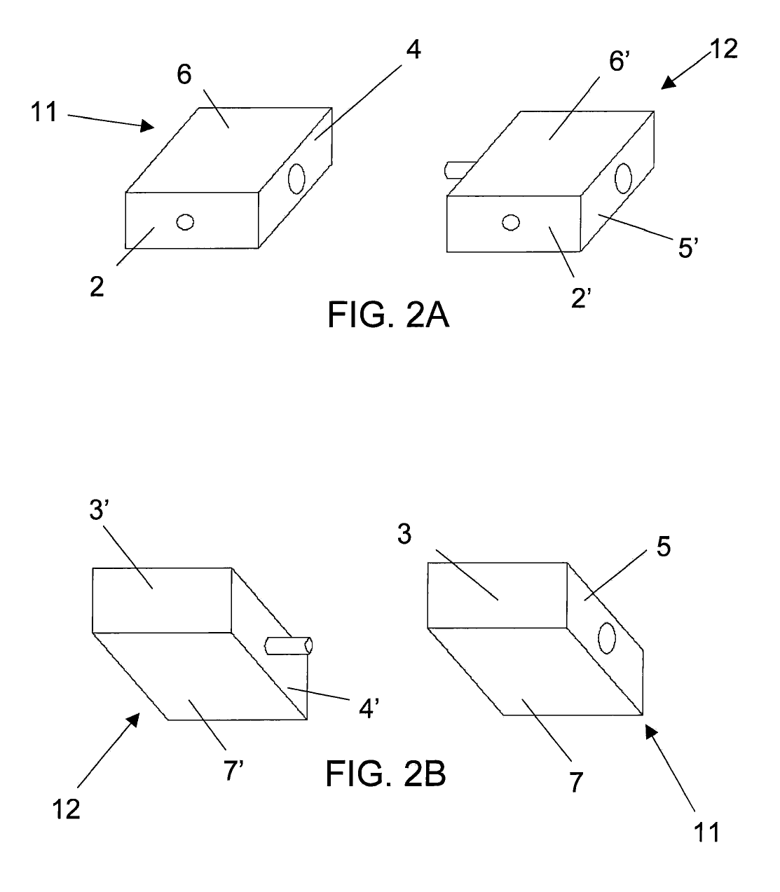

Referring to FIG. 1, in accordance with certain embodiments of the present invention, an interlocking intervertebral disc prosthesis 10 is shown. Disc prosthesis 10 comprises a left prosthetic body 11 and a right prosthetic body 12. Prosthetic bodies 11 and 12 each comprise at least one rigid endplate. In FIG. 1, each of the prosthetic bodies 11 and 12 comprise a single rigid endplate 110 and 120, respectively, but could comprise more than one endplate each. Referring to FIG. 2A, a left prosthetic body 11 has a posterior surface 2, a medial surface 4, and a superior surface 6. A right prosthetic body 12 has a posterior surface 2′, a lateral surface 5′, and a superior surface 6′. Additionally, as shown in FIG. 2B, left prosthetic body 11 has an anterior surface 3, a lateral surface 5, and an inferior surface 7. Right prosthetic body 12 has an anterior surface 3′, a medial surface 4′, and an inferior surface 7′.

In preferred embodiments, the medial surfaces of the left and right prosthetic bodies communicate with one another to interlock the prosthetic bodies. The left and right prosthetic bodies of a disc prosthesis are capable of assuming a separated configuration where they are not in physical communication with each other, as shown in FIG. 1, and are capable of assuming an interlocked configuration where they are connected to each other as shown in FIG. 4. As such, the left and right bodies can include any mutual means which allow for such an interlocking connection. In particular, any interlocking means is suitable so long as the disc prosthesis comprises a separate left prosthetic body and a separate right prosthetic body that can be connected to one another to form a single unitary device during implantation in the intervertebral space when the two are compressed medially towards one another. Non-limiting examples of interlocking means include press fit dowel pins and recesses to accept the pins or other interference fit mechanisms, various interlocking geometries, magnets, a male-female interlocking mechanism and suitable combinations. Additionally, the interlocking means may be a combination of any of the mechanisms mentioned above or any other means known in the art that are suitable for securing the left and right prosthetic bodies to one another when compressed towards one another in the medial direction. For example, FIG. 1 illustrates a male-female interlocking mechanism for the left and right prosthetic bodies. In particular, right prosthetic body 12 includes a male fastener 123 and left prosthetic body 11 includes a female fastener 113. Of course, the male fastener could be located on the left prosthetic body and the female fastener could be located on the right prosthetic body and that is true of all the embodiments described herein. Further, more than one male and female fastener could be employed on each prosthetic body, and each prosthetic body may have both male and female fasteners.

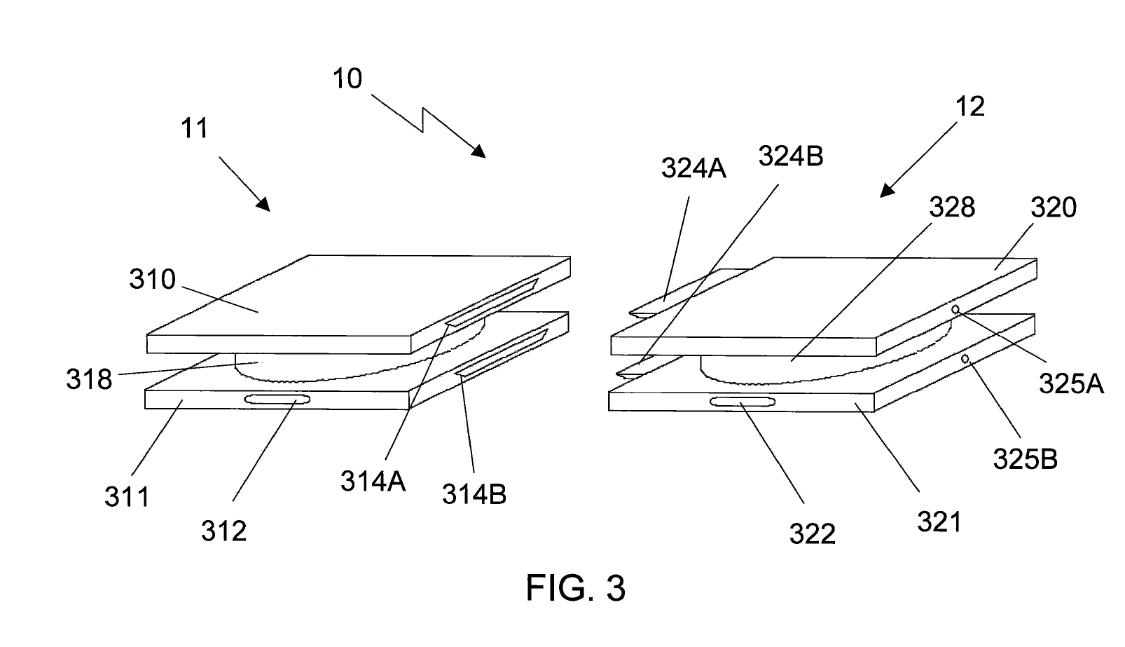

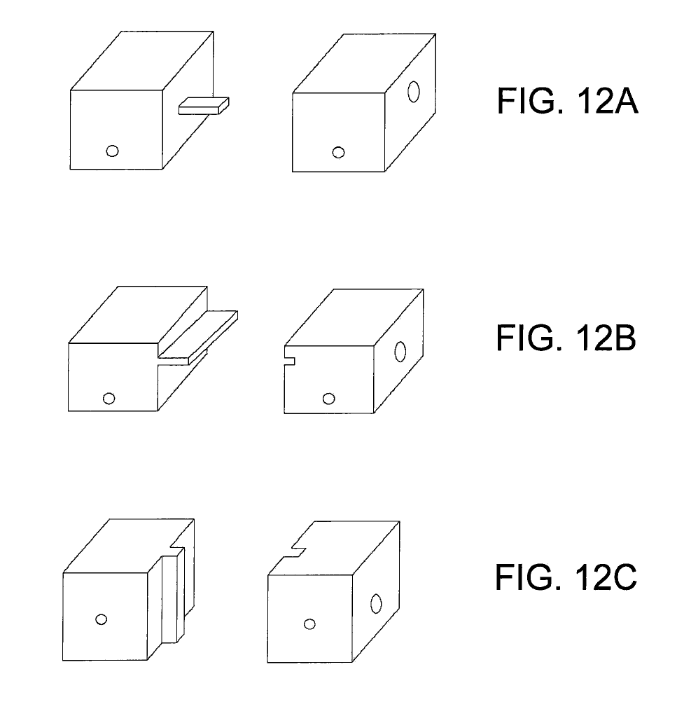

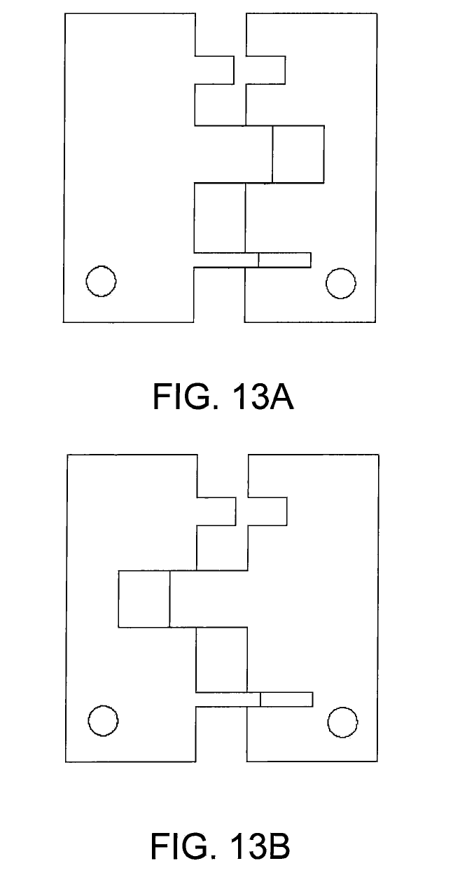

Further exemplary details regarding a male/female interlocking geometry are now provided. The male fastener may comprise any configuration known in the art to be appropriately describable as a male fastener or male connector, including, but not limited to, plugs, prongs, and convex surfaces of various configurations. Other non-limiting examples of such configurations include pin 123 shown in FIG. 1, trapezoidal shapes 324 shown in FIG. 3, and protrusions of various other configurations, such as the rectangular protrusions shown in FIG. 12A. Additionally, the male fasteners may comprise a lip that extend along the entire length of the medial surface of the prosthetic body in the posterior-anterior direction (shown in FIG. 12B), the superior-inferior direction (shown in FIG. 12C), or a diagonal configuration (not pictured). Additionally, the medial surface of the prosthetic body may comprise multiple male fasteners of various configurations, as shown in FIG. 13A.

The female fastener may comprise any configuration known in the art to be appropriately describable as a female fastener or female connector, including, but not limited to, holes, apertures, slots, channels, grooves, concave surfaces, similarly recessed geometries of various configurations and suitable combinations thereof. The female fastener may comprise any configuration capable of receiving and interlocking with a male fastener. Non-limiting examples are provided in FIGS. 12B, 12C, 13A, and 13B. In certain embodiments, the medial surface of a prosthetic body may comprise both male and female fasteners of various configurations, such as those in the example shown in FIG. 13B, which shows a series of male and female fasteners.

In certain embodiments of the present invention, the interlocking means comprise one or more protrusions located on the medial surface of the left prosthetic body, the right prosthetic body, or both, and one or more corresponding recesses for accepting the protrusions located on the medial surface of the left prosthetic body, the right prosthetic body, or both. Referring to FIG. 3, in certain embodiments of the present invention, recesses 314 are located on the medial surface of left prosthetic body 11, and protrusions 324 are located on the medial surface of right prosthetic body 12. The protrusions may have a wide range of configurations, including, but not limited to, cylinders, rectangles, dimples, spheres, hemispheres, or the trapezoidal shape shown in FIG. 3. In other embodiments, the protrusions may take the shape of a pin, such as protrusion 123 in FIG. 1. Similarly, the recesses, which accept the protrusions, may have an equally broad range of configurations. Any configuration is possible so long as the geometric shapes are able to interlock when the prosthetic bodies are compressed toward one another.

In accordance with certain embodiments, the intervertebral prosthesis may additionally comprise means for accepting an insertion tool (hereinafter referred to as the “accepting means”) located on a surface of the left prosthetic body and a surface of the right prosthetic body. For example, referring to FIG. 3, accepting means 312 may be located on the posterior surface of left prosthetic body 11 and accepting means 322 may be located on the posterior surface of right prosthetic body 12. Accepting means may be any means known in the art suitable for receiving the distal end of a surgical insertion tool in order to impact the prosthetic body into the appropriate position in the intervertebral disc space. Accepting means include (but are not limited to) one or more holes, grooves, channels, slots, any other type of recess of appropriate configuration for removable attachment to the distal end of an insertion tool, or suitable combinations thereof. The accepting means may also be one or more protrusions appropriately configured for removable attachment to the distal end of an insertion tool. The protrusions or recesses may be threaded, as appropriate, or may include the use of magnets to secure the prosthetic bodies to an insertion tool. Referring again to FIG. 1, in certain embodiments, the accepting means are recesses 112 and 122 located on the posterior surfaces (2 and 2′ in FIG. 2A) of the left and right prosthetic bodies. In other embodiments, the accepting means may be located on anterior surface 3 and 3′ of the left and right prosthetic bodies. In still other embodiments, the accepting means may be located on lateral surfaces 5 and 5′ of the left and right prosthetic bodies. The accepting means may be a combination of any of the means mentioned above or any other means known in the art that have suitable configurations for receiving the distal end of a surgical insertion tool in order to impact the prosthetic body into the appropriate position in the intervertebral disc space.

In certain embodiments, the intervertebral disc prosthesis may also comprise means for receiving the distal ends of a compression tool (hereinafter referred to as the “receiving means”). Exemplary receiving means are shown in FIG. 3 and indicated by character reference 325. In certain embodiments, receiving means 325 are located on the lateral surfaces of the left and right prosthetic bodies, as shown in FIG. 3 (receiving means for left prosthetic body 11 not shown). The receiving means may be any means known in the art suitable for receiving the distal ends of a compression tool, such as compression forceps, the compression tool being used to medially compress together two or more prosthetic bodies positioned within the intervertebral disc space, thereby engaging the interlocking means and connecting the prosthetic bodies to one another. Non-limiting examples of suitable receiving means include one or more holes, grooves, channels, slots, any other type of recess of appropriate configuration for removable attachment to the distal ends of a compression tool, and suitable combinations thereof. Similar to the accepting means above, the receiving means may also be one or more protrusions appropriately configured for removable attachment to the distal ends of a compression tool. The protrusions or recesses may include the use of magnets to secure the prosthetic bodies to a compression tool prior to compression. Referring again to FIG. 1, in certain embodiments, the receiving means are a single recess 125 located on the lateral surfaces (5 and 5′ in FIG. 2A and 2B) of the left and right prosthetic bodies. The receiving means may be a combination of any of the means mentioned above or any other means known in the art and suitable for receiving the distal ends of a compression tool, the tool being used to medially compress together two or more prosthetic bodies positioned within the intervertebral disc, thereby engaging the interlocking means and connecting the prosthetic bodies to one another.

Referring again to FIG. 3, in accordance with certain embodiments, left prosthetic body 11 may comprise an upper rigid endplate 310, a lower rigid endplate 311, and an inner core 318 disposed between upper and lower endplates 310 and 311. In these embodiments, right prosthetic body 12 may comprise an upper rigid endplates 320, a lower rigid endplate 321, and an inner core 328 disposed between the upper and lower endplates 320 and 321.

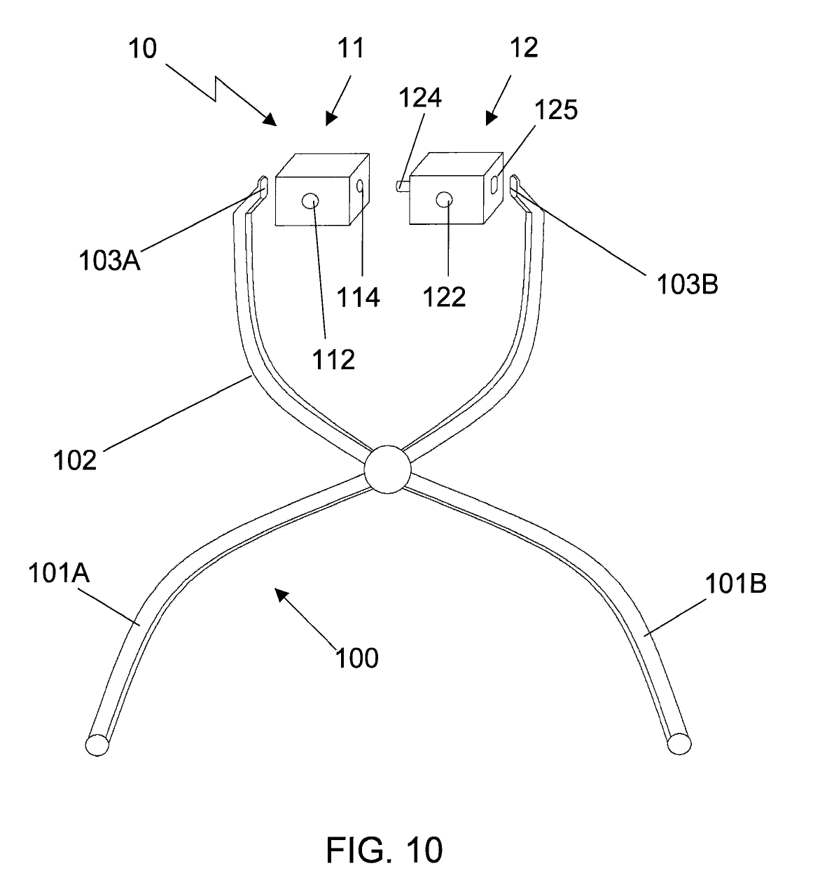

In certain other embodiments, the prosthetic bodies of the present invention comprise two rigid endplates without an inner core. The endplates of these embodiments can be similar to the endplates 20 and 40 described in FIGS. 10 through 13 of U.S. Patent Application Publication No. 2008/0051902, filed on Aug. 8, 2007 (incorporated herein by reference in its entirety).

The endplates may be fabricated from any suitable rigid biocompatible sterile material known in the art, including, but not limited to, metals, shape memory alloys, ceramic materials, polymeric materials, or any combination thereof. Non-limiting examples of suitable metallic materials include titanium, stainless steel, and cobalt chromium alloys. Non-limiting examples of suitable ceramic materials include zicronium oxide, aluminum oxide, and sintered silicon nitride. Non-limiting examples of suitable polymeric materials include polyarylesterketones, including polyetheretherketone (PEEK) and polyetherketoneketone (PEKK). The polymeric materials may also be reinforced with fillers or fibers, or may be oriented to provide additional mechanical properties. For example, the polymeric material can be reinforced with bioceramic or biolgass particles such as hydroxyaptite, which act as bioactive, bony in-growth agents and provide a reservoir of calcium and phosphate ions.

As used in the context of the rigid endplates of the present invention, “rigid” describes any material having a relatively high resilience or a modulus of elasticity greater than approximately 2.0 GPa. In certain embodiments, the modulus of elasticity is greater than 3.0 GPa. This is in contrast to the prosthesis described in U.S. Pat. No. 6,419,704 issued to Ferree (incorporated herein by reference in its entirety), which describes a body that is fillable or expandable. In U.S. Pat. No. 6,419,704, the body is composed of materials with relatively low resilience or modulus of elasticity, such as silicone and rubber. It should be noted that the entire endplates of the present invention are rigid and do not contain any non-rigid layer such as the fiber reinforced layer described in U.S. Pat. No. 6,419,704 at column 5, lines 37-39. The descriptions in this paragraph merely attempt to further clarify what is meant by the term “rigid,” and, of course, these attempts should not be interpreted in a way that would exclude any of the non-limiting examples of metals, alloys, and polymers provided throughout this specification.

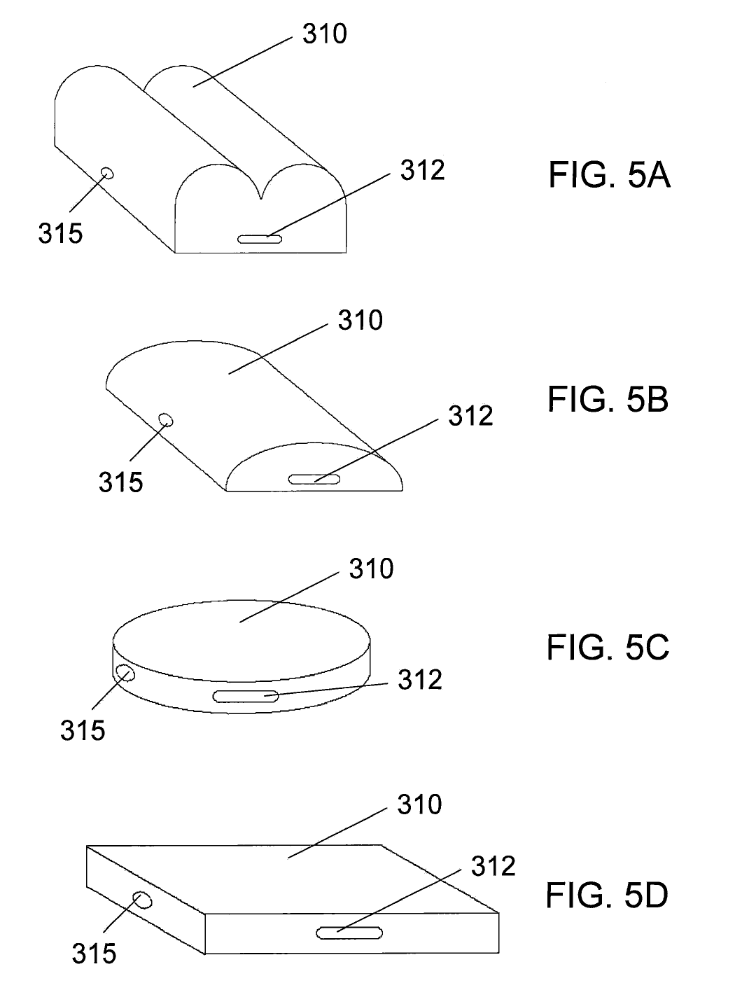

The endplates are adapted to replace the removed disc and can have any suitable configuration that allow the endplates to fit within the intervertebral space at a given spinal level, such as at the sacral, lumber, thoracic or cervical level. The endplates can also match the shape and contour of the superior or inferior surfaces of adjacent vertebral bodies to better mate against the vertebral bodies. Referring again to FIG. 3, for example, upper endplates 310 and 320 may have a planar superior surface that allows for more optimal or extended surface area contact with the adjacent porous or cancellous inferior surface of an adjacent upper vertebral body. Similarly, lower endplates 311 and 321 may have an inferior surface that allows for similar contact with the superior surface of an adjacent lower vertebral body. FIGS. 5A through 5D illustrate several possible configurations for the endplates. In each of FIGS. 5A through 5D, upper endplate 310 of left prosthetic body 11 is shown for demonstrative purposes. It should be noted that any of the profiles depicted or described herein are equally applicable to any of the endplates. It should also be noted that the placement of recess 312 (configured to accept the distal end of an insertion tool) and recess 315 (configured to accept a distal end of a compression tool) are similarly demonstrative and do not represent the placement of these elements in all embodiments of the present invention.

The endplates may have any of a broad variety of configurations, including, but not limited to, an arcuate profile (FIG. 5A), a domed or convex-like profile (FIG. 5B), a cylindrical profile (FIG. 5C), or a rectangular profile (FIG. 5D). The endplates may have a tapered thickness that increases in the anterior to posterior direction to provide an anterior to posterior lordotic taper to better restore the natural curvature of the spine. In certain embodiments, the endplates may be configured to at least partially surround an inner core, forming a complete prosthetic body. These profiles are merely exemplary of the plurality of configurations the endplates may assume, and any other configuration suitable for use as an intervertebral disc replacement may be used. Any combination of suitable profiles may be used, and each endplate need not have the same configuration. In addition, any of the endplate configurations described in U.S. Patent Application Publication No. 2008/0051902 cited above may be suitable for use with the present invention.

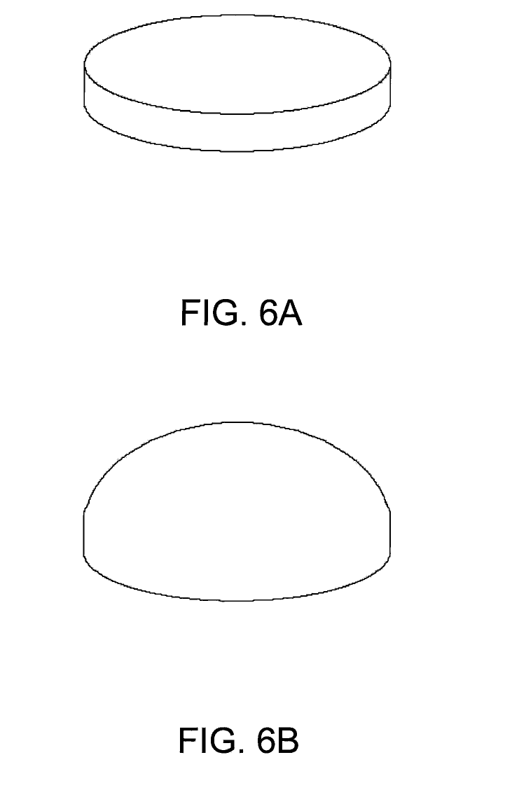

When used, the inner core may comprise any suitable biocompatible sterile material including, but not limited to, various polymers and plastics (such as polyethylene), metals and alloys, and ceramic materials. Preferably, the inner core comprises a somewhat flexible material, such as an elastomeric material, allowing it to mimic the functionality of the nucleus pulposus of a natural intervertebral disc. Similar to the endplates described above, the inner core may comprise any configuration that allows it to be at least partially contained between the endplates. Non-limiting examples of inner core profiles include cylindrical (FIG. 6A), spherical or hemi-spherical (FIG. 6B), or any of the profiles suitable for the endplates described above.

In certain embodiments, the endplates and the inner core may comprise the same material. These elements need not be comprised of the same material however, and each endplate and inner core may comprise any of the materials mentioned above, independent of the composition of any other endplate or core.

In certain embodiments, the prosthesis may further comprise means for securing the prosthesis to adjacent superior and inferior vertebrae (hereinafter referred to as the “securing means”). Returning to FIGS. 2A and 2B, the securing means may be located on superior surfaces 6 and 6′, inferior surfaces 7 and 7′, or both the superior and inferior surfaces. Additionally, the securing means may be located on the left prosthetic body, right prosthetic body, or both. The securing means is configured to anchor the prosthesis in the intervertebral space and to prevent unwanted shifting of the prosthesis after surgery. Accordingly, the securing means may comprise any configuration that achieves this goal, including, but not limited to, a rough or jagged surface, the anchors described in U.S. Patent Application Publication No. 2008/0051902, various biocompatible adhesives such as bone cement, and suitable combinations thereof.

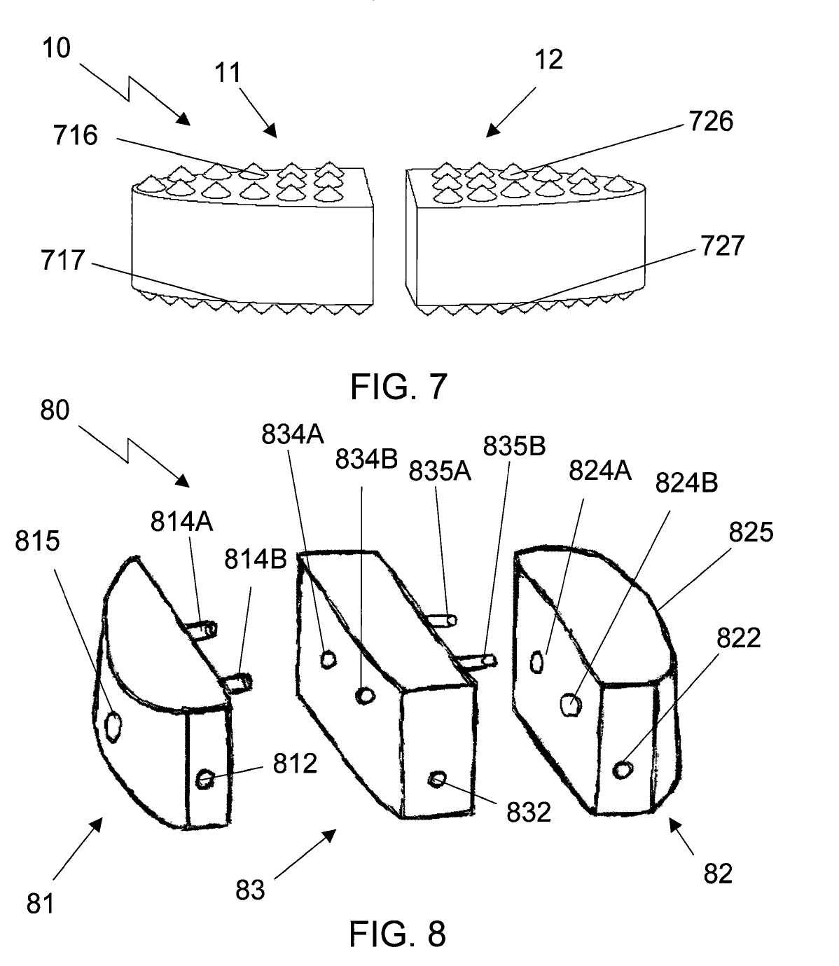

Referring to FIG. 7, in certain embodiments, the securing means comprises a plurality of serrations 716, 717, 726, and 727 located on both the superior and inferior surfaces of left and right prosthetic bodies 11 and 12.

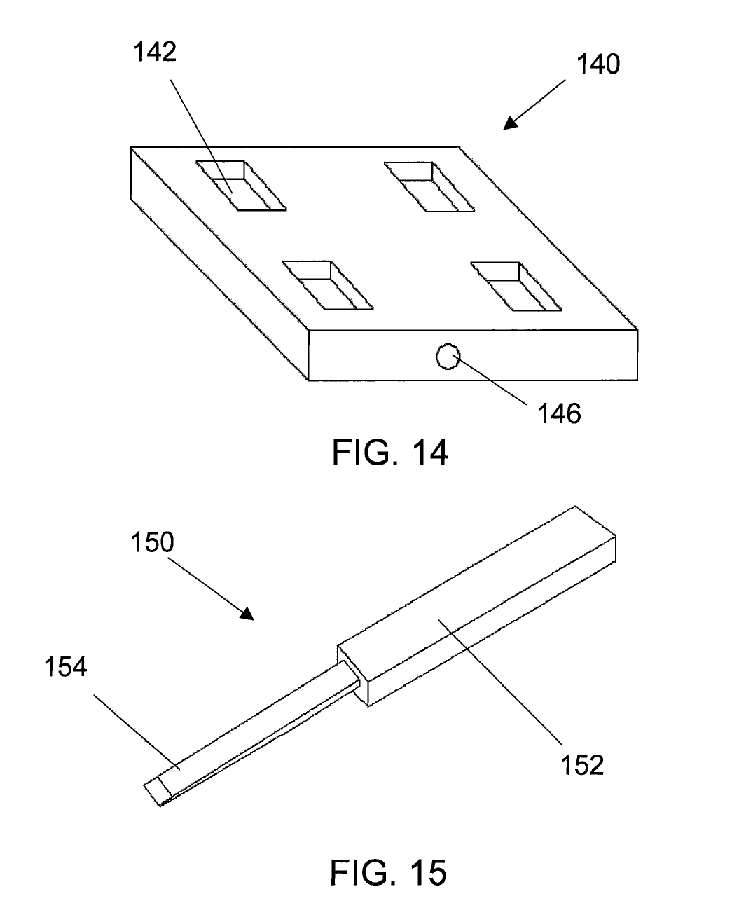

In certain embodiments, the endplates of the left and right prosthetic bodies comprise pores extending from the superior surface to the inferior surface of the prosthetic bodies. These embodiments may be used to revise a disc prosthesis to an interbody fusion cage. Referring to FIG. 14, an exemplary endplate 140 is shown having pores 142 and accepting means 146. The pores may comprise a variety of configurations and function much like pores 72 in FIG. 1 of U.S. Patent Application Publication No. 2008/0051902 cited above. The pores need not all assume the same configuration. Although rectangular pores are shown in FIG. 14, the pores may comprise any other configuration known to those skilled in the art as suitable for use in an interbody fusion cage. These pores accommodate bone in-growth to provide solid fixation of the prostheses. In certain embodiments, the superior and inferior surfaces of the endplates of the left and right prosthetic bodies include a porous coating or osteoconductive mesh structure. Alternatively, the surfaces can be made porous, such as by titanium plasma spray. For example, the endplates of the left and right prosthetic bodies may comprise a titanium bead coating applied onto their respective superior and inferior surfaces via spraying or sintering. Alternatively, the superior or inferior surfaces of the plates can be roughened in order to promote bone in-growth into the defined roughened surfaces of the disc prosthesis.

In embodiments where the endplate assemblies are fenestrated and thus are capable of being revised to an interbody fusion cage, the present invention provides for kits comprising endplate assemblies as generally described above and at least one, and preferably two, interbody fusion cages. The spinal fusion cage comprises a cage body and at least one projection extending from a surface of the cage body. The interbody cage can be inserted between the superior and inferior endplate assemblies, replacing the inner cores used in certain other embodiments. The interbody fusion cage can be any type known in the art such as, for example, a vertical fusion cage (such as a Harms cage) and a rectangular fusion cage (such as a Brantigan cage). Further descriptions of converting a disc replacement to an interbody fusion cage are described in U.S. Patent Application Publication No. 2008/0051902 cited above.

The porous layer or surface on the left and/or right prosthetic body may also deliver desired pharmacological agents. The pharmacological agent may be, for example, a growth factor to assist in the repair of the vertebral endplates and/or the annulus fibrosis. Non-limiting examples of growth factors include a bone morphogenetic protein, transforming growth factor (TGF-β), insulin-like growth factor, platelet-derived growth factor, fibrolast growth factor, or other similar growth factor or combinations thereof having the ability to repair the endplates an/or the annulus fibrosis of an intervertebral disc.

In certain embodiments, the disc prosthesis can be fenestrated, if it is desired, for example, to revise the disc prosthesis to an interbody fusion cage. In such embodiments, the present invention provides a kit comprising a disc prosthesis and at least one, and preferably two, interbody fusion cages. A disc prosthesis in accordance with these embodiments would preferably comprise a left and right prosthetic body, each prosthetic body having at least two rigid endplates The interbody cage or cages can be inserted between the endplates of the left and right prosthetic bodies, replacing the inner cores used in certain other embodiments. The interbody fusion cage can be any type known in the art such as, for example, a vertical fusion cage (such as a Harms cage) and a rectangular fusion cage (such as a Brantigan cage).

The present invention additionally provides an interlocking intervertebral disc prosthesis comprising three or more prosthetic bodies. An example of a prosthesis in accordance with this embodiment comprises a left prosthetic body comprising at least one rigid endplate, a right prosthetic body comprising at least one rigid endplate, and at least one additional prosthetic body comprising at least one endplate. The endplate of the at least one additional prosthetic body is preferably rigid, as that term is described above. Each of the left and right prosthetic bodies has a medial surface, a lateral surface, a posterior surface, an anterior surface, a superior surface, and an inferior surface; and the at least one additional prosthetic body has a left surface, a right surface, a posterior surface, an anterior surface, a superior surface, and an inferior surface. A prosthesis of this embodiment additionally comprises means for interlocking the prosthetic bodies located on the medial surfaces of the left and right prosthetic bodies and the left and right surfaces of the at least one additional prosthetic body. In this embodiment, the prosthesis further comprises means for accepting an insertion tool located on a surfaces of each of the prosthetic bodies. In certain other embodiments, the prosthesis may additionally comprise means for accepting the distal ends of a compression tool located on the lateral surfaces of each of the left and right prosthetic bodies.

FIG. 8 depicts an example of certain embodiments of the present invention having left and right prosthetic bodies 81 and 82 and a single additional prosthetic body 83. In certain embodiments, prosthetic bodies 81, 82, and 83 each comprise means 814, 824, 834, and 835 for interlocking the prosthetic bodies and means 812, 822, and 832 for accepting an insertion tool. Left prosthetic body 81 and right prosthetic body 82 each further comprise means for receiving the distal ends of a compression tool 815 and 825 (not shown). The interlocking means, accepting means, and receiving means are all identical to the respective means described for the two-body prosthesis above but of course could be different. Additional body 83's interlocking means 834 and 835 are located on the left and right surfaces of body 83 and are configured to engage with the interlocking means of an adjacent body, regardless of whether that body is a left prosthetic body, a right prosthetic body, or another prosthetic body. While a three-body prosthesis is shown in FIG. 8, in accordance with certain embodiments of the present invention, any number of bodies may be impacted into the intervertebral disc space and compressed together limited only by the dimensional parameters of the intervertebral space.

The present invention also provides a surgical kit for replacing an intervertebral disc with an interlocking intervertebral disc prosthesis. The kit comprises an interlocking intervertebral disc prosthesis, an insertion tool attachable to each of the prosthetic bodies of the intervertebral disc prosthesis, and a compression tool. More than one insertion tool can be provided. An insertion tool comprises an attachment means for attaching one end of the insertion tool to the accepting means on a prosthetic body, and the compression tool comprises attachments means for attaching to the receiving means on a prosthetic body.

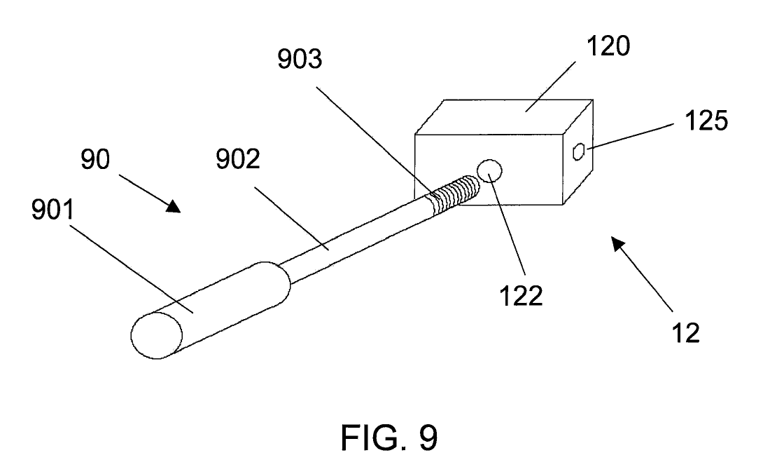

Referring to FIG. 9, in certain embodiments, an insertion tool 90 comprises an elongated member 902 having attachment means 903 located at a distal end. In certain embodiments, the insertion tool 90 also comprises a handle 901. The attachment means may comprise any structure known in the art that may removably attach to accepting means 122 on the prosthetic bodies. In certain embodiments, attachment means 903 comprises external threads configured to threadedly engage with internal threads on accepting means 122 of an exemplary prosthetic body. However, threads are not required for all embodiments, and other means for removably attaching an insertion tool to a prosthetic body may be used, such as interference fits or magnets.

Referring to FIG. 10, a surgical kit may further comprise a compression tool, such as a pair of compression forceps 100. The compression forceps 100 may comprise handles 101 (comprised of individual members 101A and 101B) pivotably attached to a pair of tongs 102 that are in a moveable relationship relative to each other. The compression forceps are arranged such that when a user squeezes handles 101A and 101B, tongs 102 move towards one another medially. The tongs may comprise means 103 for connecting to prosthetic bodies 11 and 12 located at the distal-most ends of the tongs. Means 103 may be any configuration that can engage receiving means 115 and 125 on prosthetic bodies 11 and 12.

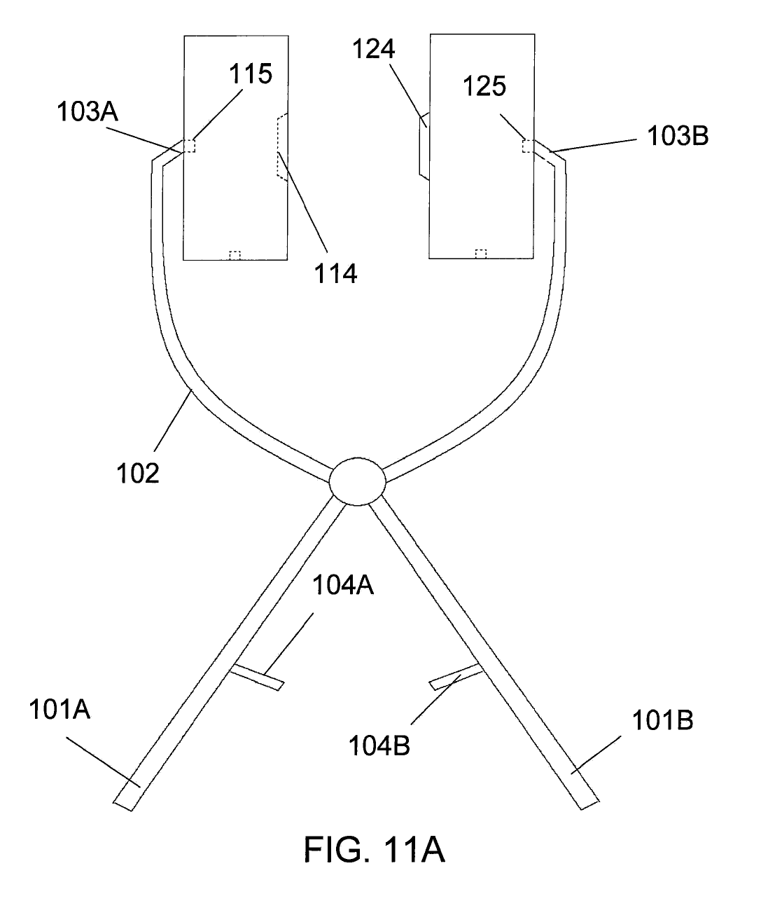

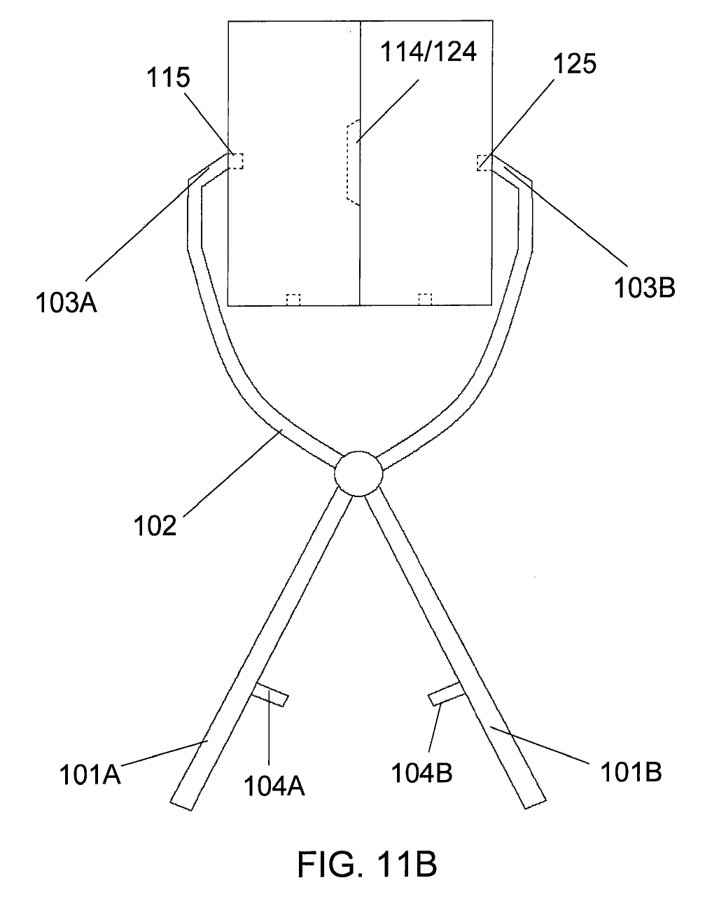

Referring to FIGS. 11A and 11B, forceps 100 are used to compress prosthetic bodies 11 and 12 towards one another medially in the intervertebral disc space. When a user squeezes handles 101A and 101B of forceps 100, prosthetic bodies 11 and 12 move from an open, separated configuration shown in FIG. 11A to a closed, interlocked position shown in FIG. 11B. When the prosthetic bodies are compressed towards one another, interlocking means 114 and 124 engage, interlocking left prosthetic body 11 to right prosthetic body 12.

In certain embodiments, the compression forceps may further comprise a safety stop 104 that prevents the forceps from closing past a certain point during use. The purpose of the safety stop is to prevent the forceps' tongs or handles from squeezing the sensitive spinal organs such as the dural tube. In certain embodiments, the safety stop comprises a protrusion extending laterally in a direction substantially perpendicular to the handles. In certain embodiments, the protrusion may be on both sides of the compression forceps. In certain embodiments, the total length of the protrusions is greater than the diameter of the dural tube. In this way, the safety stop will ensure that neither the handles nor the tongs will squeeze the dural tube. Although safety stop 104 is shown on handles 101A and 101B in FIGS. 11A and 11B, it may also be placed elsewhere on the compression forceps, such as the tongs.

In accordance with certain embodiments, the kit may further comprise a plurality of box chisels used to form entry paths for the interlocking disc prosthesis. The box chisels may be of varying sizes suitable for creating an entry path into the intervertebral disc space. An example of a box chisel 150 in accordance with certain embodiments of the present invention is shown in FIG. 15, having an elongated member 152 and chisel head 154. The chisel head may assume any configuration known by those skilled in the art as suitable for creating an entry path to a patient's intervertebral disc space.

One of skill in the art will appreciate that each of the interlocking means, accepting means, and receiving means described above may serve multiple or dual roles. For example, in certain embodiments, the interlocking means double as the accepting means. In these embodiments, the interlocking means, which interlock the left and right prosthetic bodies to one another, additionally accept the distal ends of insertion tools. Similarly, in other embodiments, the accepting means double as receiving means. In these embodiments, the accepting means, which accept distal ends of insertion tools, additionally receive the distal ends of compression tools used to compress the left and right prosthetic bodies medially toward one another. In still other embodiments, the insertion tools and compression tools are one in the same such that one tool is used to insert and compress the prosthetic bodies. In still other embodiments, the receiving means double as the accepting means. In these embodiments, the receiving means, which receive distal ends of compression tools, additionally accept the distal ends of insertion tools. Further, with the exception of the interlocking means which are located on the medial surfaces of the prosthetic bodies in most embodiments, the accepting and receiving means can be located on the anterior, posterior and/or lateral surfaces of the prosthetic bodies. Although the above shared configurations are possible in various embodiments and methods of the present invention, for purposes of clarity the means are described separately in the discussion of exemplary surgical procedures below.

The present invention also provides a method for surgically implanting an interlocking intervertebral disc prosthesis in an intervertebral space of a patient. Such a method comprises removing the natural intervertebral disc from the intervertebral space and inserting a multi-piece prosthetic into the intervertebral space. The multi-piece prosthetic comprises a left prosthetic body comprising at least one rigid endplate and a separate right prosthetic body comprising at least one rigid endplate. The two prosthetic bodies are inserted into the intervertebral space as two separate components (i.e. in an non-interlocked configuration). The method further comprises interlocking the left prosthetic body and the right prosthetic body in the intervertebral space.

A non-limiting example of a process for inserting an intervertebral disc prosthesis will now be described. The patient is placed in the prone position on a standard radiolucent operative table. In certain embodiments, the surgical approach is a posterior approach to the spine. In other embodiments, the patient is placed in the supine position, and the approach may be anterior or lateral, for example. The dura matter on either the left or the right lateral side of the spinal canal is retracted to expose the intervertebral disc space, and the intervertebral disc on that side is removed. One or more appropriately sized box chisels are then used to form an entry path for the intervertebral disc prosthesis. The dura matter on the opposing lateral side of the spinal canal is then retracted to expose the opposing lateral intervertebral disc space. Again, the intervertebral disc on that side is removed, and one or more box chisels are used to form an entry path for the intervertebral disc prosthesis. Once the natural intervertebral disc is removed, each of a plurality of interlocking intervertebral disc prosthetic bodies is impacted individually into the intervertebral space using a plurality of insertion tools. Once all of the prosthetic bodies have been impacted into the appropriate positions, each of the insertion tools is removed from each of the prosthetic bodies. A pair of compression forceps is attached to the lateral sides of the rightmost lateral prosthetic body and the leftmost lateral prosthetic body. The compression forceps are squeezed together, attaching each of the plurality of interlocking intervertebral disc prosthetic bodies to one another by engaging the interlocking means on each prosthetic body. While a posterior approach has been described, in accordance with certain other embodiments, the interlocking intervertebral disc prosthesis may also be implanted through an anterior or lateral approach.

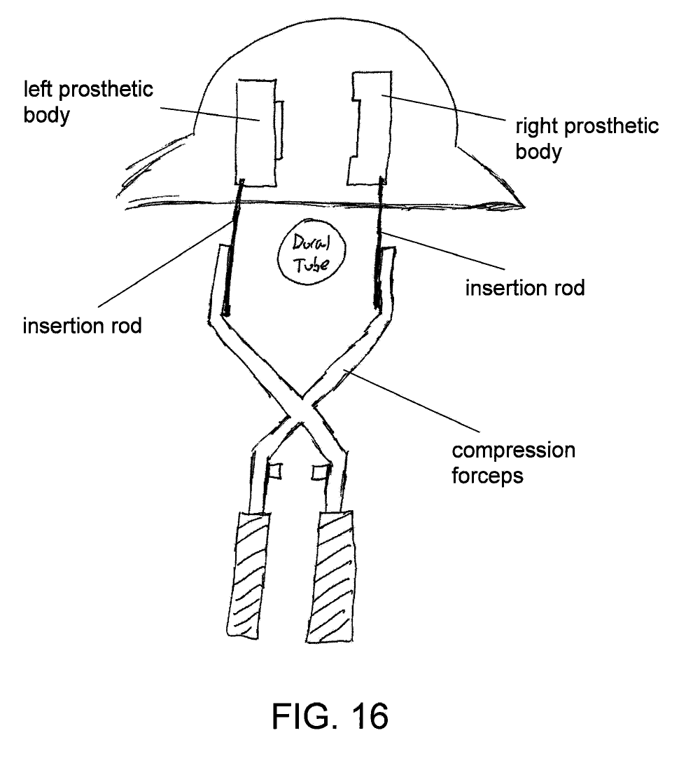

In other embodiments, a patient's natural vertebral disc may be removed and a plurality of intervertebral disc prosthetic bodies may be implanted into the patient as described above. In the methods of these embodiments, the insertion tools are not removed from the prosthetic bodies, and a compression tool (such as compression forceps) are attached to the leftmost lateral insertion tool and the rightmost lateral insertion tool (as shown in FIG. 16). The compression tool is then squeezed together, attaching each of the plurality of interlocking intervertebral disc prosthetic bodies to one another by engagement of the interlocking means on each prosthetic body. Unlike the previously described embodiment, in such an embodiment, the compression forceps are never in direct communication with the prosthetic bodies during compression of the forceps. Accordingly, in such an embodiment, the prosthetic bodies need not have means for receiving the distal ends of compression tools located on the lateral surfaces of the left and right prosthetic bodies since the compression tool compresses the prosthetic bodies by way of the insertion tools attached to the prosthetic bodies. While a posterior approach has been described, in accordance with certain other embodiments, the interlocking intervertebral disc prosthesis may also be implanted through an anterior or lateral approach.

While various embodiments have been described, other embodiments are plausible. It should be understood that the foregoing descriptions of various examples of an interlocking intervertebral disc prosthesis are not intended to be limiting, and any number of modifications, combinations, and alternatives of the examples may be employed to facilitate the effectiveness of replacing a natural intervertebral disc with a low-profile disc prosthesis requiring relatively minimally invasive surgery.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. An interlocking intervertebral disc prosthesis, comprising:
a left prosthetic body comprising at least one rigid endplate;
a right prosthetic body comprising at least one rigid endplate, each of the prosthetic bodies having a lateral surface, a medial surface, a posterior surface, an anterior surface, a superior surface, and an inferior surface;
means for interlocking the left prosthetic body to the right prosthetic body, the interlocking means located on the medial surfaces of the prosthetic bodies; and
means for accepting an insertion tool located on a surface of the left prosthetic body and a surface of the right prosthetic body.

2. The prosthesis of claim 1, further comprising means for receiving the distal ends of compression tools located on the lateral surfaces of the left and right prosthetic bodies.

3. The prosthesis of claim 1, further comprising means for receiving the distal ends of compression tools located on the posterior surfaces of the left and right prosthetic bodies.

4. The prosthesis of claim 1, further comprising means for receiving the distal ends of compression tools located on the anterior surfaces of the left and right prosthetic bodies.

5. The prosthesis of claim 1, wherein the means for accepting an insertion tool are located on the posterior surfaces of the right and left prosthetic bodies.

6. The prosthesis of claim 1, wherein the means for accepting an insertion tool are located on the lateral surfaces of the right and left prosthetic bodies.

7. The prosthesis of claim 1, wherein the means for accepting an insertion tool are located on the anterior surfaces of the right and left prosthetic bodies.

8. The prosthesis of claim 1, wherein the interlocking means comprise a male and female fastening system, the male fastener located on the medial surface of the left prosthetic body and the female fastener located on the medial surface of the right prosthetic body.

9. The prosthesis of claim 1, wherein the interlocking means comprise a male and female fastening system, the male fastener located on the medial surface of the right prosthetic body and the female fastener located on the medial surface of the left prosthetic body.

10. The prosthesis of claim 1, wherein the interlocking means comprise one or more magnets to interlock located on the medial surface of the right prosthetic body, the left prosthetic body, or both.

11. The prosthesis of claim 1, wherein each of the prosthetic bodies comprises two rigid endplates and an inner core disposed between the two rigid endplates.

12. The prosthesis of claim 1, further comprising means for securing the prosthesis to adjacent superior and inferior vertebrae, the securing means located on the inferior surface, the superior surface, or both the inferior and superior surfaces of the left prosthetic body, the right prosthetic body, or both the left and right prosthetic bodies.

13. The prosthesis of claim 1, further comprising a plurality of serrations to secure the prosthesis to adjacent superior and inferior vertebrae, the plurality of serrations located on the inferior surface, the superior surface, or both the inferior and superior surfaces of the left prosthetic body, the right prosthetic body, or both the left and right prosthetic bodies.

14. The prosthesis of claim 1, further comprising one or more keels to secure the prosthesis to adjacent superior and inferior vertebrae, the one or more keels located on the inferior surface, the superior surface, or both the inferior and superior surfaces of the left prosthetic body, the right prosthetic body, or both the left and right prosthetic bodies.

15. The prosthesis of claim 1, further comprising pores extending from the superior surface to the inferior surface of the at least one endplate of the left and right prosthetic bodies.

16. The prosthesis of claim 1, wherein each of the left and right prosthetic bodies further comprises a porous layer coating the superior and inferior surfaces of the prosthetic bodies.

17. The prosthesis of claim 2, wherein the compression tools comprise a pair of compression forceps.

18. An interlocking intervertebral disc prosthesis, comprising:
a left prosthetic body comprising at least one rigid endplate;
a right prosthetic body comprising at least one rigid endplate, each of the prosthetic bodies having a lateral surface, a medial surface, a posterior surface, an anterior surface, a superior surface, and an inferior surface;
at least one male fastener located on the medial surface of one of the left prosthetic body and the right prosthetic body and at least one female fastener located on the medial surface of the other of the left prosthetic body and the right prosthetic body;
wherein the male and female fasteners are configured and positioned such that the left prosthetic body and the right prosthetic body interlock in an applied position; and
one or more openings for accepting an insertion tool located on a surface of the left prosthetic body and a surface of the right prosthetic body.

19. The prosthesis of claim 18, further comprising one or more apertures for receiving the distal ends of a compression tool located on the lateral surfaces of the left and right prosthetic bodies.

20. The prosthesis of claim 18, wherein the at least one male fastener comprises one or more protrusions located on the medial surface of one of the left prosthetic body and the right prosthetic body and wherein the at least one female fastener comprises one or more corresponding recesses located on the medial surface of the other of the left prosthetic body and the right prosthetic body.

21. An interlocking intervertebral disc prosthesis, comprising:
a left prosthetic body comprising at least one rigid endplate;
a right prosthetic body comprising at least one rigid endplate, wherein each of the left and right prosthetic bodies has a medial surface, a lateral surface, a posterior surface, an anterior surface, a superior surface, and an inferior surface;
at least one additional prosthetic body comprising at least one endplate, the at least one additional prosthetic body having a left surface, a right surface, a posterior surface, an anterior surface, a superior surface, and an inferior surface;
means for interlocking the at least one additional prosthetic body to the left and right prosthetic bodies or any additional prosthetic bodies positionable between the left and right prosthetic bodies, the interlocking means located on the medial surfaces of the left and right prosthetic bodies and the left and right surface of the at least one additional prosthetic body; and
means for accepting an insertion tool located on a surface of each of the prosthetic bodies.

22. The prosthesis of claim 21, further comprising means for receiving the distal ends of a compression tool located on the lateral surfaces of the left and right prosthetic bodies.

23. A kit for replacing an intervertebral disc with an interlocking intervertebral disc prosthesis, comprising:
the interlocking disc prosthesis of claim 1;
a plurality of insertion tools attachable to the prosthetic bodies, each insertion tool comprising an elongated member; and
a compression tool comprising a set of handles and a pair of tongs pivotably attached to the set of handles.

24. The kit of claim 23, further comprising a plurality of box chisels each having a different configuration to form entry paths for insertion of the disc prosthesis.

25. The kit of claim 23, wherein the compression tool comprises a pair of compression forceps.

26. The kit of claim 25, wherein the compression forceps further comprise a safety stop, wherein the safety stop comprises means for preventing the compression forceps from closing past a certain point during use.

27. The kit of claim 25, wherein the compression forceps further comprise a safety stop, the safety stop comprising one or more protrusions on each side of the forceps, the protrusions extending towards one another in the lateral direction.

28. A kit for converting an interlocking intervertebral disc prosthesis to an interbody fusion device, comprising:
the interlocking disc prosthesis of claim 1;
a plurality of insertion tools attachable to the prosthetic bodies, each insertion tool comprising an elongated member; and
at least one interbody fusion cage.

29. A method for surgically implanting an interlocking intervertebral disc prosthesis in an intervertebral space of a patient, the method comprising:
removing the natural intervertebral disc from the intervertebral space;
inserting a multi-piece prosthetic into the intervertebral space, the multi-piece prosthetic comprising a left prosthetic body comprising at least one rigid endplate and a right prosthetic body comprising at least one rigid endplate; and
interlocking the left prosthetic body and the right prosthetic body.

30. A method for surgically implanting an interlocking intervertebral disc prosthesis in an intervertebral space of a patient, the method comprising:
removing the intervertebral disc on the right lateral side and the left lateral side of the intervertebral space;
impacting a left and a right intervertebral disc prosthetic bodies individually in the intervertebral space, each of the prosthetic bodies having a lateral side;
attaching a pair of compression forceps to the lateral sides of the right and left intervertebral disc prosthetic bodies; and
squeezing the compression forceps to attach the left and right intervertebral disc prosthetic bodies to one another.

31. A method for surgically implanting an interlocking intervertebral disc prosthesis in an intervertebral space of a patient, the method comprising:
removing the intervertebral disc on the right lateral side and the left lateral side of the intervertebral space;
impacting a left and a right intervertebral disc prosthetic bodies individually in the intervertebral space using a left and right insertion tool, each of the prosthetic bodies having a lateral side;
attaching a pair of compression forceps to the lateral sides of the right and left insertion tools; and
squeezing the compression forceps to attach the left and right intervertebral disc prosthetic bodies to one another.

32. An interlocking intervertebral disc prosthesis, comprising:
a left prosthetic body comprising at least one rigid endplate;
a right prosthetic body comprising at least one rigid endplate, each of the prosthetic bodies having a lateral surface, a medial surface, a posterior surface, an anterior surface, a superior surface, and an inferior surface; and
means for interlocking the left prosthetic body to the right prosthetic body and accepting an insertion tool, the interlocking and accepting means located on the medial surfaces of the prosthetic bodies.
